(19) United States
(12) Patent Application Publication
(10) Pub. No.: US 2024/0269221 A1
(43) Pub. Date: Aug. 15, 2024

(54) A SYNERGISTIC PHYTOEXTRACT FORMULATION FOR ANTI-INFLAMMATORY ACTION

(71) Applicants: Prashanth VARKEY, Kerala Thrissur (IN); Rosemary PRASHANTH, Kerala Thrissur (IN)

(72) Inventors: Prashanth VARKEY, Kerala Thrissur (IN); Rosemary PRASHANTH, Kerala Thrissur (IN)

(21) Appl. No.: 18/570,097

(22) PCT Filed: Aug. 18, 2021
(86) PCT No.: PCT/IB2021/057580

§ 371 Date: Dec. 14, 2023

(30) Foreign Application Priority Data
Jun. 14, 2021 (IN) 202141026454

(51) Int. Cl. A61K 36/906; A61K 9/48; A61K 31/352; A61K 31/4525; A61K 36/537; A61K 36/67

(52) U.S. Cl. CPC A61K 36/906; A61K 9/4825; A61K 31/352; A61K 31/4525; A61K 36/537; A61K 36/67

(57) ABSTRACT

A synergistic phytoextract formulation for anti-inflammatory action is disclosed. The synergistic phytoextract formulation comprises effective amount of phytoextracts selected from the group consisting of Elettaria cardamomum (cardamomum) in the range of 40% by weight, Salvia rosmarinus (rosemary) in the range of 40% by weight and piperine extract in the range 2% by weight. The active molecule 1, 8 Cineol from the Elettaria cardamomum (cardamomum) and Salvia rosmarinus (rosemary) exhibits excellent muscle relaxant effects by decreasing smooth muscle contractions of airways, anti-inflammatory action by blocking cytokines release and analgesic effects. The synergistic phytoextract formulation is formulated for oral administration in the forms of capsules, microcapsules or tablets.

### EARLIEST PRIORITY DATE

This Application claims priority from a patent application filed in India having Patent Application No. 202141026454, filed on Jun. 14, 2021 and titled “A SYNERGISTIC PHYTOEXTRACT FORMULATION FOR ANTI-INFLAMMATORY ACTION.”

### FIELD OF THE INVENTION

Embodiments of a present disclosure relates to a herbal composition. More particularly, the present disclosure relates to a synergistic phytoextract formulation for anti-inflammatory action in patients recovering from Covid-19 disease.

### BACKGROUND OF THE INVENTION

It is known that coronavirus disease (COVID-19) is an infectious disease caused by a newly discovered coronavirus. The disease emerged first in December 2019 in Wuhan. Hubei, China. This deadly disease rapidly spread to almost 188 countries around the world, with a mortality rate of approximately 5%. The outbreak was declared a Public health Emergency of International Concern by the World Health Organization (WHO) in January 2020 and was recognized as a pandemic in March 2020.

A novel coronavirus, (SARS-CoV), was identified as the etiological agent of SARS. SARS-CoV is an enveloped, positive-strand RNA virus. Its large RNA genome is approximately 30000 nucleotides in length and encodes a nonstructural replicase complex and structural proteins, including spike (S), envelope (E), membrane (M) and nucleocapsid (N) proteins. Spike protein is the envelope protein responsible for invasion of host cells.

It has been found that COVID-19 positive individual may remain asymptomatic or show symptoms that may be mild, moderate or severe in nature. Most common symptoms of COVID-19 positive range from fever, dry cough, tiredness, etc., ranging to less common symptoms such as body aches and pains, sore throat, diarrhea, conjunctivitis, severe headache and loss of taste or smell and appearance of rash on skin, or discoloration of fingers or toes. The disease COVID-19 can spread from person to person through small droplets from the nose or mouth land on objects and surfaces around the person which are spread when a person with COVID-19 coughs or exhales.

Apart from the these, substantial portion of the population also suffer from a range of post Covid disease complications i.e., Covid-19 symptoms can persist for months after a person contracts the virus. The virus can damage the lungs, heart and brain, which increases the risk of long-term health problems. The type of pneumonia often associated with Covid-19 can cause long-standing damage to the tiny air sacs (alveoli) in the lungs. The resulting scar tissue can lead to long-term breathing problems.

As a result, it may be noted that the post-Covid syndrome is a new unique phenomenon, which demands to be addressed. Its symptoms, particularly the extreme fatigue, can have huge downstream effects on a person's quality of life—impacting family life and work productivity.

US20190062785 A1 discloses methods and compositions for inducing an immune response that confers dual protection against infections by either or both of a rabies virus and a coronavirus, and/or which can be used therapeutically for an existing infection with rabies virus and/or a coronavirus to treat at least one symptom thereof and/or to neutralize or clear the infecting agents. In particular, the present disclosure provides a recombinant rabies virus vector comprising a nucleotide sequence encoding at least one coronavirus immunogenic glycoprotein fragment, as well as pharmaceutical compositions comprising the vaccine vectors.

KR101128088 B1 discloses a composition for the prevention or treatment of coronavirus infection and the composition for the inhibition of 3CL protease activity, including the buckthorn extract or Wakame tree extract, buckwheat extract or fractions thereof, Wakame tree extract or fractions thereof In addition, the quinomethed triterpene compounds extracted from these compounds exhibit the effect of inhibiting the activity of the 3CL protease and simultaneously show the effect of inhibiting virucidal and cytopathic activity against various coronaviruses, and thus can be useful for the prevention or treatment of coronavirus infection.

WO2020086759A2 discloses methods of use and pharmaceutical liquid compositions that are orally administered to the lungs through vaporization and aerosol generating devices providing multifunctional treatment for lung and respiratory diseases, wherein the composition comprises of at least one plant extract, thiol amino acid, vitamin, chelating agent and antioxidant.

CN109641022 discloses method for reducing oxidative stress using a composition comprising of encapsulated cannabinoid compounds and terpene, to cause reduction in oxidative stress.

Therefore, there is a need for developing a natural herbal formulation with anti-inflammatory action for use in Covid-19 patients to shorten the time of recovery with no chemical induced harmful side effects.

### OBJECTS OF THE INVENTION

It is an object of the present invention to provide a synergistic phytoextract formulation comprising of natural herbs for treatment of post Covid-19 complications.

It is an objective of the present invention to provide a synergistic phytoextract formulation which is stable, safe, and economical for recovery of Covid-19 disease.

It is another objective of the present invention to provide anti-infective, anti-inflammatory and prevent damage to body cells caused by Covid-19 disease.

It is still another objective of the present invention to reduce the potential for cytokines to occur and induce anti-inflammatory action.

It is another objective of the present invention to provide a synergistic formulation that is capable of being administered orally.

Furthermore, the objects and advantages of the present invention will become apparent from the following description below.

### SUMMARY OF THE INVENTION

In accordance with an embodiment of the present invention, a synergistic phytoextract formulation for anti-inflammatory action is disclosed, wherein the synergistic phytoextract formulation for anti-inflammatory action comprises of an effective amount of phytoextracts extracted from Elettaria cardamomum in the range of 40% by weight, Salvia rosmarinus in the range of 40% by weight and piperine extract in the range 2% by weight.

In accordance with an embodiment of the present invention, the piperine extract is obtained from Piper nigrum and Piper longum.

In accordance with an embodiment of the present invention, the phytoextracts of Elettaria cardamomum and Salvia Rosmarinus contains active molecule 1,8 Cineol.

In accordance with an embodiment of the present invention, the active molecule 1,8 Cineol exhibits excellent muscle relaxant effects by decreasing smooth muscle contractions of airways, anti-inflammatory action by blocking cytokines release and analgesic effects.

In accordance with an embodiment of the present invention, wherein the piperine extract boosts bioavailability.

In accordance with an embodiment of the present invention, wherein the synergistic phytoextract formulation is formulated for oral administration in the form of capsules, microcapsules, or tablets.

In accordance with an embodiment of the present invention, wherein the synergistic phytoextract formulation is effective in inducing anti-inflammatory action.

In accordance with an embodiment of the present invention, wherein the phytoextract formulation acts as an anti-oxidant and exhibits anti-viral and anti-bacterial properties.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### DETAILED DESCRIPTION OF THE INVENTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

As used herein “synergistic” means interaction or combination of two or more components, substances, ingredients to produce a combined effect greater than the sum of their separate effects.

As used herein “Post-Covid Syndrome” means Covid-19 symptoms which persist for months, particularly the extreme fatigue and other related symptoms which exerts tremendous downstream effects on a person's quality of life-impacting family life and work productivity.

As used herein “Elettaria cardamomum” commonly known as green or true cardamom, is a herbaceous, perennial plant in the ginger family, native to southern India, wherein Cardamom is the dried fruit of Elettaria cardamomum.

As used herein “Salvia rosmarinus” commonly known as rosemary, is a generally erect, rounded, evergreen shrub with aromatic, needle-like, gray-green leaves and tiny, two-lipped, pale blue to white flowers.

As used herein “Piperine extract” refers to a standardized extract containing the active alkaloid piperine, derived from the fruit of the plant Piper nigrum (black pepper) and/or the plant Piper longum L. (long pepper), with thermogenic properties and also acts as a bioavailability enhancer.

According to an embodiment of the present invention, a synergistic phytoextract formulation for anti-inflammatory action is disclosed, wherein the synergistic phytoextract formulation for anti-inflammatory action comprises of an effective amount of phytoextracts extracted from Elettaria cardamomum in the range of 40% by weight, Salvia rosmarinus in the range of 40% by weight and piperine extract in the range 2% by weight.

According to an embodiment of the present invention, the piperine extract is obtained from Piper nigrum and Piper longum.

According to an embodiment of the present invention, the phytoextracts of Elettaria cardamomum and Salvia Rosmarinus contains active molecule 1,8 Cineol.

According to an embodiment of the present invention, the active molecule 1,8 Cineol exhibits excellent muscle relaxant effects by decreasing smooth muscle contractions of airways, anti-inflammatory action by blocking cytokines release and analgesic effects.

According to an embodiment of the present invention, wherein the piperine extract boosts bioavailability.

According to an embodiment of the present invention, wherein the synergistic phytoextract formulation is formulated for oral administration in the form of capsules, microcapsules, or tablets.

According to an embodiment of the present invention, wherein the synergistic phytoextract formulation is effective in inducing anti-inflammatory action.

According to an embodiment of the present invention, wherein the phytoextract formulation acts as an anti-oxidant and exhibits anti-viral and anti-bacterial properties.

According to an embodiment of the present invention, the synergistic phytoextract formulation acts as a natural herbal anti-inflammatory formulation for use in Covid-19 patients for shortening their time of recovery with no chemical induced side effects.

According to an embodiment of the present invention, the synergistic phytoextract formulation improves recovery performance and endurance for normal physical activities and reduces stress on body systems resulting from Covid-19 disease.

### Example Method of Preparation of the Synergistic Phytoextract Formulation

According to an embodiment of the present invention, a 500 mg hard gel capsules are formulated using weight by percentage ratios of Elettaria cardamomum (Cardamom extract) 40%, Salvia Rosmarinus (Rosemary extract) 40% and Piperine extract 2%. These extracts are obtained through at least one of the methods of extraction such as steam distillation, solvent extraction, hydro-distillation, microwave extraction, etc., to meet a significantly high content of 1,8 cineole. The ratios of each extract as mentioned above are measured and blended in by mechanical stirring. The combined formulation is then microencapsulated using maltodextrin to generate a powder form which is then filled in hard gel capsules.

According to an embodiment of the present invention, the synergistic formulation is purely natural with intended for use as in oral administration, wherein the formulation comprises of extracts of Elettaria cardamomum, Salvia rosmarinus and piperine extract with significant quantity of active molecule 1,8 Cineol along with other anti-oxidants, anti-viral and antibacterial phytoconstituents.

According to an embodiment of the present invention, the synergistic phytoextract formulation relieves various pathophysiological conditions of Covid-19 and reduces the progressive symptoms post recovery. Among the constituents within the formulation, the extracts of Elettaria cardamomum (Cardamom) Salvia Rosmarinus (Rosemary) contain the active molecule 1,8 Cineol which exhibits excellent muscle relaxant effects by decreasing smooth muscle contractions of airways, anti-inflammatory (by blocking cytokines release) and analgesic effects with anti-viral against Covid-19 virus. Further, the piperine extract boosts the bioavailability and the favourable safety and efficacy profile of 1,8 Cineol from the natural phytoextracts such as Elettaria cardamomum (Cardamom) Salvia Rosmarinus (Rosemary) adds to the benefit of its use.

Overall, this synergistic phytoextract formulation in a form of capsule/tablets makes oral consumption easy and feasible for daily administration.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A synergistic phytoextract formulation for anti-inflammatory action, comprising an effective amount of phytoextracts extracted from Elettaria cardamomum in the range of 40% by weight, Salvia rosmarinus in the range of 40% by weight and piperine extract in the range 2% by weight.

2. The synergistic phytoextract formulation as claimed in claim 1, wherein the piperine extract is obtained from Piper nigrum and Piper longum.

3. The synergistic phytoextract formulation as claimed in claim 1, wherein the phytoextracts of Elettaria cardamomum and Salvia Rosmarinus contains active molecule 1,8 Cineol.

4. The synergistic phytoextract formulation as claimed in claim 1, wherein the active molecule 1,8 Cineol exhibits excellent muscle relaxant effects by decreasing smooth muscle contractions of airways, anti-inflammatory action by blocking cytokines release and analgesic effects.

5. The synergistic phytoextract formulation as claimed in claim 1, wherein the piperine extract boosts bioavailability.

6. The synergistic phytoextract formulation as claimed in claim 1, wherein the synergistic phytoextract formulation is formulated for oral administration in the form of capsules, microcapsules, or tablets.

7. The synergistic phytoextract formulation as claimed in claim 1, wherein the phytoextract formulation is effective in inducing anti-inflammatory action.

8. The synergistic phytoextract formulation as claimed in claim 1, wherein the phytoextract formulation acts as an anti-oxidant and exhibits anti-viral and anti-bacterial properties.
